(19) United States
(12) Patent Application Publication
(10) Pub. No.: US 2014/0107639 A1
(43) Pub. Date: Apr. 17, 2014

(54) RENAL DENERVATION SYSTEM AND METHOD

(71) Applicants: Yongxing Zhang, Irvine, CA (US); Cary Hata, Irvine, CA (US); Alan De La Rama, Cerritos, CA (US)

(72) Inventors: Yongxing Zhang, Irvine, CA (US); Cary Hata, Irvine, CA (US); Alan De La Rama, Cerritos, CA (US)

(73) Assignee: St. Jude Medical Cardiology Division, Inc., St. Paul, MN (US)

(21) Appl. No.: 14/124,419

(22) PCT Filed: Jun. 6, 2012
(86) PCT No.: PCT/US2012/041029

§ 371 Date: Dec. 6, 2013

Related U.S. Application Data
(60) Provisional application No. 61/493,849, filed on Jun. 6, 2011.

(51) Int. Cl. A61B 18/14

(52) U.S. Cl. CPC A61B 18/1492
USPC 606/33

(57) ABSTRACT

A method for denervation comprises introducing a distal portion of a catheter through an interior of a vessel of a patient to a location at or proximate one of a renal pelvis or a calyx. The catheter includes an elongated catheter body extending longitudinally between a proximal end and a distal end. The catheter body includes the distal portion at the distal end and a catheter lumen from the proximal end to the distal end. Energy is delivered from the distal portion to cause renal denervation, for example, by denervating at least some tissue proximate at least one of the renal pelvis or a renal vessel from a location at or proximate the renal pelvis or the calyx from a location at or proximate the calyx.

### RELATED APPLICATIONS

This application is based on and claims the benefit of International Application Number PCT/US2012/041029, published as WO 2012/170482 and filed on Jun. 6, 2012, and U.S. Provisional Application No. 61/493,849, filed on Jun. 6, 2011, the entire disclosures of which are incorporated herein by reference.

### BACKGROUND OF THE INVENTION

The present invention relates to renal denervation for the treatment of hypertension, other cardiovascular disorders, and chronic renal diseases.

Hypertension is a major public health problem due to its high prevalence in the population. Prevalence of hypertension worldwide was estimated as high as 26.4% in 2000. By 2026 it is expected to affect 29.2% of the world's adult population, increasing the number of hypertensive adults by 60% and reaching 1.5 billion patients worldwide. Hypertension is considered a major risk factor for cardiovascular disease. High blood pressure exerts deleterious effects on the vasculature throughout the body promoting atherosclerosis and arteriosclerosis. Hypertension is associated with increased rates of heart disease (myocardial infarction, heart failure, left ventricular hypertrophy, arrhythmias), brain disease (stroke, ischemic attack), renal disease (chronic renal failure, microalbuminuria), and eye disease (hypertensive retinopathy).

Kidneys plays a key role in initiation and maintenance of hypertension, particularly with increased renal sympathetic activation. In experimental functional studies, it has been shown that renal sympathetic nerve activation enhances the spillover of norepinephrine, while renal denervation results in a marked decrease of norepinephrine. Denervation is defined herein as partially or totally blocking nerve conduction. Denervation may be achieved by high-frequency stimulating, or overstimulating, or ablating the nerves. Renal denervation diminishes or reduces renal sympathetic nerve activity, increases renal blood flow (RBF), and decreases renal plasma norepinephrine (NE) content. Renal denervation abolishes the induction of renin release by conditions that are known to stimulate renin release such as volume depletion, head-up tilt, or reduced renal perfusion pressure. Renal sympathetic nerve activation induces sodium reabsorption, while significant sodium excretion occurs with renal denervation. Indeed, increased or decreased sodium intake induces a response of renal sympathetic nerves towards the opposite direction, resulting in rapid changes of urinary sodium excretion. The effects of renal denervation on renal blood flow (RBF) were demonstrated in rabbits, where RBF was greater than 50% in denervated compared to innervated kidneys, one week after renal denervation.

Clinical studies have shown the effectiveness of RF ablation via the renal arteries for treatment of resistant hypertension for up to two years. The RF renal arterial ablation results in renal efferent and afferent denervation, and it is believed that the sustained blood pressure reduction is mainly due to afferent denervation.

The kidney is very richly innerved in all parts of the nephron and the renal vasculature. Afferent renal nerves reside mostly in the renal pelvic wall and renal afferent denervation should be effective in treatment of hypertension. Anatomically, the renal pelvis is in close proximity of the renal arterial branches reaching in kidney, as well as the renal nerves innerving the kidney along the renal arterial branches.

Heretofore, techniques for sympathetic/renal denervation have included sympathectomy, surgical renal denervation, and renal arterial ablation. Sympathectomy had been mainly applied in patients with severe or malignant hypertension, as well as in patients with cardiovascular deterioration despite relatively good blood pressure reduction by other means. After the introduction of antihypertensive drugs, sympathectomy was reserved for patients who failed to respond to antihypertensive therapy or could not tolerate it. Total sympathectomy or splanchnicectomy, an extended operation performed in the early years, was found to be impractical and poorly tolerated by patients. This was later replaced by a more conservative surgery, from the eighth to twelfth dorsal vertebra performed in one or two stages, still requiring a 2-4 weeks hospital stay and a 1-2 months recovery period, and was only performed in a few selected institutions. Adverse events were usual, annoying, and in some cases serious, and included orthostatic hypotension, orthostatic tachycardia, palpitations, breathlessness, anhidrosis, cold hands, intestinal disturbances, loss of ejaculation, sexual dissatisfaction, thoracic duct injuries and atelectasis. Surgical renal denervation is mainly an experimental technique in animal studies.

Transvascular renal arterial denervation involves delivering energy via the renal artery to damage the renal nerves in adjacent proximity. An RF renal denervation system consists primarily of a catheter and an RF generator. Thermal energy is delivered through the arterial wall to ablate the adjacent renal nerves. While renal denervation via renal arterial ablation has been effective, its limitations exist. For example, patients with certain medical conditions are not eligible for renal arterial ablation. These conditions include renal stenosis, unstable renal arterial plaque, main renal artery too short, or main renal arterial diameter too small, torturous renal arterial anatomy, history of renal interventions, and multiple main renal arteries.

### BRIEF SUMMARY OF THE INVENTION

Embodiments of the present invention are directed to transvenous renal nerve modulation apparatuses and methods for the treatment of hypertension, other cardiovascular disorders, and chronic renal diseases. It is desirable to provide selective renal denervation techniques that also have less and lower clinical risks for the treatment of hypertension and other renal and cardiovascular pathological conditions.

In accordance with an aspect of the present invention, a method for denervation comprises: introducing a distal portion of a catheter through an interior of a vessel of a patient to a location at or proximate one of a renal pelvis or a calyx, the catheter including an elongated catheter body extending longitudinally between a proximal end and a distal end, the catheter body including the distal portion at the distal end and a catheter lumen from the proximal end to the distal end; and delivering energy from the distal portion to denervate at least some tissue proximate at least one of the renal pelvis or the calyx.

In some embodiments, the distal portion of the catheter is introduced through a ureter of the patient. The distal portion of the catheter is introduced through a urethra and a bladder of the patient. The method further comprises inserting a uretheral access sheath through the urethra into the ureter to facilitate introduction of the distal portion of the catheter through the urethra and the ureter. Energy is delivered from the distal portion to cause renal afferent denervation of at least some tissue proximate at least one of the renal pelvis or the calyx. The energy delivered is selected from the group consisting of radio-frequency (RF) energy, electrical energy, laser energy, ultrasonic energy, high-intensity focused ultrasound (HIFU) energy, cryogenic energy, thermal energy, chemical energy, and mechanical energy. The method further comprises delivering a fluid via the catheter lumen through the distal portion to at least one of the renal pelvis or the calyx. The method further comprises manipulating the distal portion through a control member disposed near the proximal end of the catheter to adjust a position and an orientation of delivering energy from the distal portion. The method further comprises obtaining via the distal portion an image of denervation of the at least some tissue.

In accordance with another aspect of the invention, a method for denervation comprises: introducing a distal portion of a catheter through an interior of a vessel of a patient to a location at or proximate a renal pelvis, the catheter including an elongated catheter body extending longitudinally between a proximal end and a distal end along a longitudinal axis, the catheter body including the distal portion at the distal end and a catheter lumen from the proximal end to the distal end; and delivering energy from the distal portion at the location at or proximate the renal pelvis to cause renal denervation.

In some embodiments, energy is delivered from the distal portion at the location at or proximate the renal pelvis to cause renal denervation in at least one of the renal pelvis or a renal blood vessel. Energy is delivered from the distal portion to cause renal afferent denervation of at least some tissue proximate at least one of the renal pelvis or a renal vessel. The method further comprises delivering a fluid via the catheter lumen through the distal portion to the renal pelvis. The distal portion of the catheter is introduced through a urethra, a bladder, and a ureter of the patient.

In accordance with another aspect of this invention, a method for denervation comprises: introducing a distal portion of a catheter through an interior of a vessel of a patient to a location at or proximate a renal pelvis, the catheter including an elongated catheter body extending longitudinally between a proximal end and a distal end, the catheter body including the distal portion at the distal end and a catheter lumen from the proximal end to the distal end; and delivering energy from the distal portion at the location at or proximate the renal pelvis to denervate at least some tissue proximate the renal pelvis.

In specific embodiments, energy is delivered from the distal portion at the location at or proximate the renal pelvis to denervate at least some tissue adjacent a renal pelvis wall of the renal pelvis. Energy is delivered from the distal portion via contact between the distal portion and a renal pelvis wall of the renal pelvis. The distal portion of the catheter is introduced through a urethra, a bladder, and a ureter of the patient.

These and other features and advantages of the present invention will become apparent to those of ordinary skill in the art in view of the following detailed description of the specific embodiments.

### BRIEF DESCRIPTION OF THE DRAWINGS

FIG. 1 is a block diagram of a system for renal denervation according to an embodiment of the invention.

FIG. 2 is an elevational view of the irrigated ablation catheter.

FIG. 3 is another elevational view of the irrigated ablation catheter of FIG. 2.

FIG. 4 is a system installation diagram of an RF ablation system with an irrigated ablation catheter.

FIG. 5 is a block diagram of the RF ablation system of FIG. 4.

FIG. 6 shows an example of a distal portion of an irrigated ablation catheter.

FIG. 7 shows another example of a distal portion of a catheter in the form of an assembly of staggered ablation elements.

FIG. 8 shows a path in the form of a broken line through the urethra, bladder, and ureter to introduce the distal portion of the catheter to the renal pelvis and, in some case, to the renal calyx.

FIG. 9 shows an example of a uretheral sheath.

FIG. 10 shows an example of a flow diagram illustrating a renal denervation process according to an embodiment of the invention.

### DETAILED DESCRIPTION OF THE INVENTION

In the following detailed description of the invention, reference is made to the accompanying drawings which form a part of the disclosure, and in which are shown by way of illustration, and not of limitation, exemplary embodiments by which the invention may be practiced. In the drawings, like numerals describe substantially similar components throughout the several views. Further, it should be noted that while the detailed description provides various exemplary embodiments, as described below and as illustrated in the drawings, the present invention is not limited to the embodiments described and illustrated herein, but can extend to other embodiments, as would be known or as would become known to those skilled in the art. Reference in the specification to “one embodiment,” “this embodiment,” or “these embodiments” means that a particular feature, structure, or characteristic described in connection with the embodiment is included in at least one embodiment of the invention, and the appearances of these phrases in various places in the specification are not necessarily all referring to the same embodiment. Additionally, in the following detailed description, numerous specific details are set forth in order to provide a thorough understanding of the present invention. However, it will be apparent to one of ordinary skill in the art that these specific details may not all be needed to practice the present invention. In other circumstances, well-known structures, materials, circuits, processes and interfaces have not been described in detail, and/or may be illustrated in block diagram form, so as to not unnecessarily obscure the present invention.

In the following description, relative orientation and placement terminology, such as the terms horizontal, vertical, left, right, top and bottom, is used. It will be appreciated that these terms refer to relative directions and placement in a two dimensional layout with respect to a given orientation of the layout. For a different orientation of the layout, different relative orientation and placement terms may be used to describe the same objects or operations.

Exemplary embodiments of the invention, as will be described in greater detail below, provide system and method of renal denervation via the ureter-renal pathway for treatment of hypertension and other renal and cardiovascular related pathological conditions.

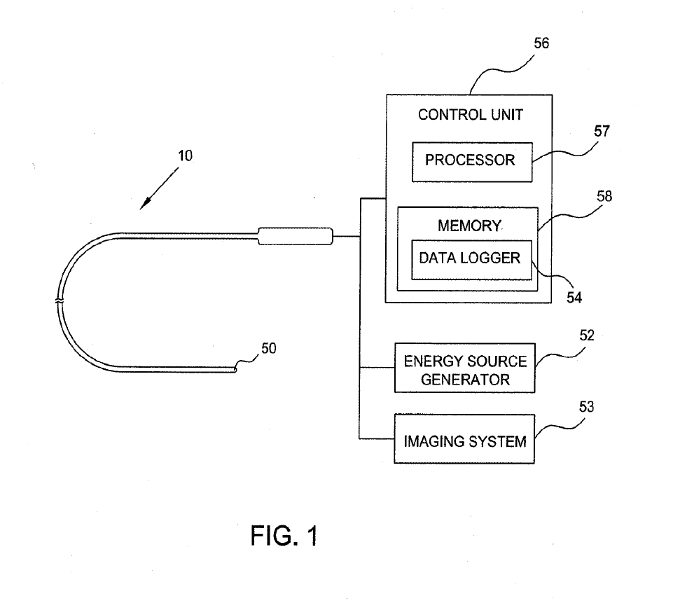

FIG. 1 is a block diagram of a system for renal denervation according to an embodiment of the invention. The system includes a catheter 10 having a distal portion 50, an energy source generator 52, and optionally a data logger 54 to display and store data during the denervation procedure. The data logger 54 may be a module in a control unit 56 which includes a processor 57 and a memory 58. For example, the data logger 54 is a software module stored in the memory 58. The distal portion 50 includes denervation element(s) and optionally an endoscope or some other type of imaging device, coupled to an imaging system 53, to provide tissue imaging capability using imaging techniques such as ultrasound or the like. The imaging capability allows the operator to verify the region being ablated/denervated, to check the progress of the ablation/denervation, and to address safety concerns.

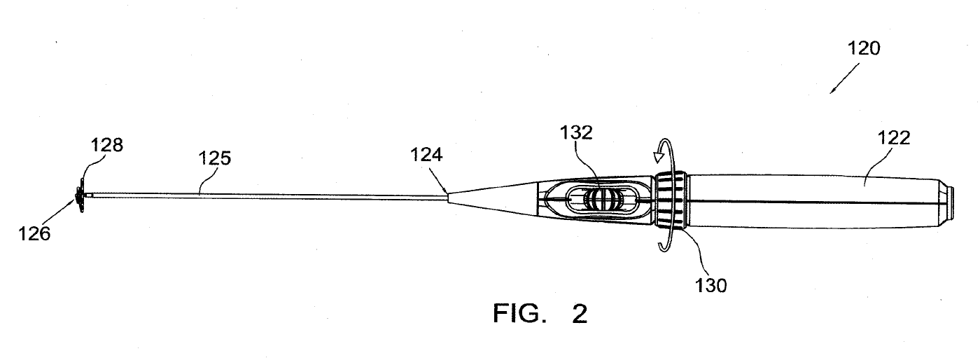

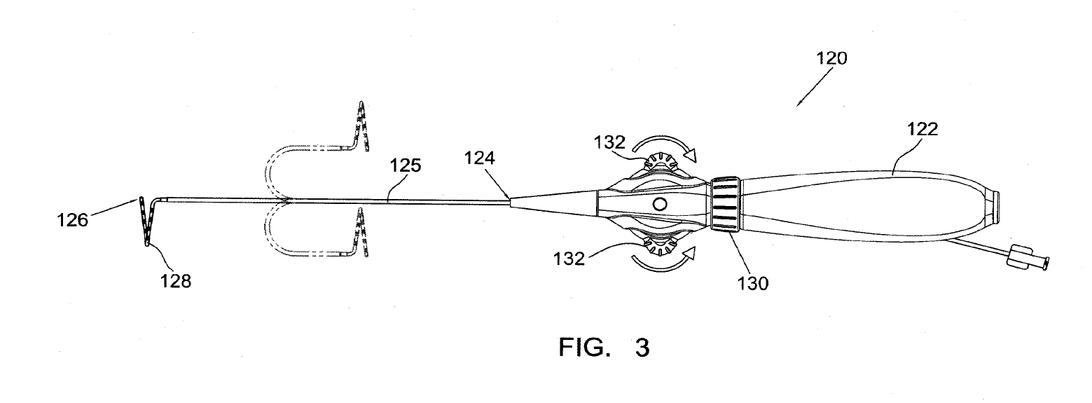

FIGS. 2 and 3 are elevational views of an irrigated ablation catheter 120 showing a handle 122 connected to a proximal end 124 of an elongated body 125 for manipulating the shape of a distal portion of the catheter 120 near a distal end 126. In FIG. 2, the distal portion of the catheter 120 includes a loop 128 having a plurality of segmented ablation electrodes separated by electrically nonconductive segments. The handle 122 includes a first roller 130 for changing the size of the loop 128, and a second set of rollers or sliders 132 for bidirectional bending of the elongated body 125. The distal tip of the catheter may be rotated up to about 360 degrees. The loop 128 is optional and can be eliminated in a different embodiment. The electrodes on the catheter can be either sensing or ablating electrodes, or both. Sensing electrodes may be used for any of a variety of sensing, including physical/mechanical, fluid dynamics, electrical, or chemical (e.g., force, flow and rate, impedance, temperature, pH values). Energy may be delivered to the ablation electrodes, simultaneously or sequentially, or selectively. For selective delivery, a clinician can select, via a user interface of the energy supply such as an RF generator, the specific electrodes to be utilized in the denervation/ablation process.

The catheter shaft 125 may be made of silicone, polyurethane (PU), Pebax, or a combination of PU and silicone, or some other biocompatible polymers and/or metallic materials. The catheter body 125 typically has a size smaller than about 10 Fr which is sufficiently large to house an imaging device such as an endoscope as well as components for ablation/denervation. In specific embodiments, the catheter body preferably has a size smaller than about 5 Fr, which will be safer and easier to use for access through the urethra and ureter, for example. The specification of a current flexible ureteroscope can be used as a guide in configuring the catheter: tip diameter of 6.9-9.8 F (7.5 F most common), optics composed of fiber optic bundles, single working channel of 3.6 F, access by guidewire (0.035 in Nitinol or 0.038 in stainless steel), and average accessory length of 100 cm.

The catheter body 125 may have a lumen for stylet delivery or an open lumen through the catheter body for over the wire (OTW) delivery. An open lumen can also be used for delivering saline irrigation to cool the electrodes, and/or delivering chemical agents, and/or injecting contrast media for renal arterial/venous grams. In addition, irrigation holes can be located at the distal end 126 and/or at or near the electrodes, or along the catheter body 125 for egress of irrigation fluid. Irrigation of the catheter can be automatic or manual. Automatic irrigation is achieved with a pump, while manual irrigation requires the saline to be hand injected to flush the catheter during ablation. The purpose of the irrigation is for more effective lesion while mitigating the damage, for example, of the endothelium and the endo-layer of the renal pelvis or some other target region for ablation/denervation. The lumen for irrigation and contrast media may be shared with a switchable valve. Such a valve is preferably operated manually by the clinician performing the ablation procedure. In some embodiments, the catheter can have multiple lumens, for the purposes of irrigation, contrast media injection, optical coherence tomography (OCT), ultrasound probe insertion, endoscope, chemical agents delivery, or other mechanisms of imaging and treatment instrumentations. The lumen can be shared for the applications stated. The catheter may also have the feature of delivering chemical agents, including denervation agents, either via an open lumen directly or by using micro-needle techniques.

In different embodiments, the catheter may be steerable uni-directionally or bi-directionally deflectable. The catheter shaft may have variable diameters allowing in some embodiments for the distal portion to be less rigid (i.e., more compliant or deformable) than the proximal sections. The deflection capability of the catheter can be of the range from about 0 to 90 degrees, with the deflection of the distal portion in the range of about 4 mm to 15 mm. The distal portion of the catheter may have a passive deflection curve with various shapes in both 2D and 3D configurations. The catheter tip may include a radiopaque portion to facilitate viewing under fluoroscopy. The catheter may include a lumen for optical fiber access and/or a lumen for ultrasound probe access.

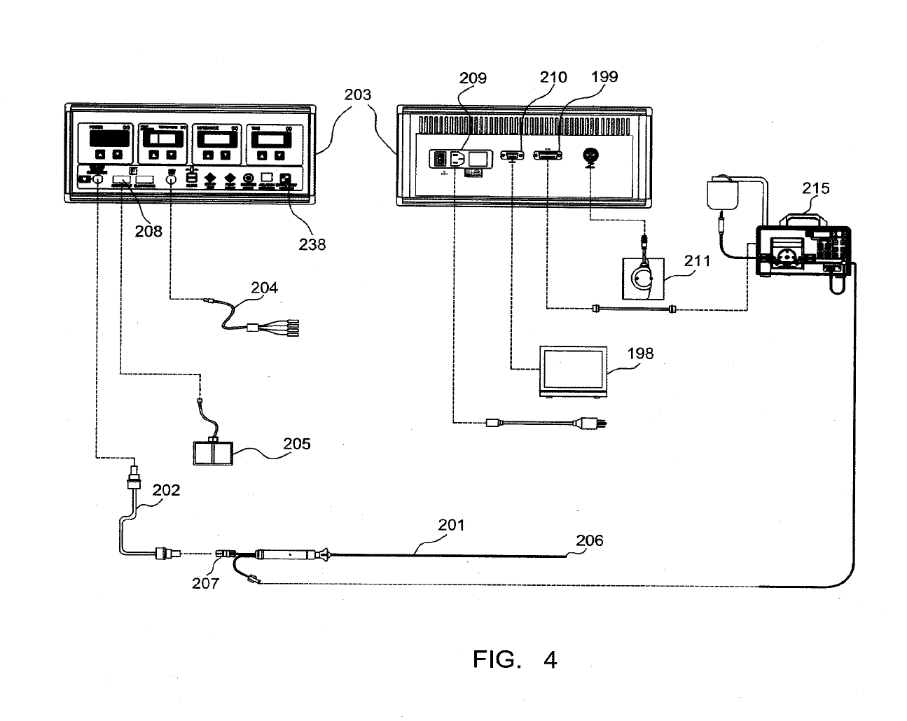

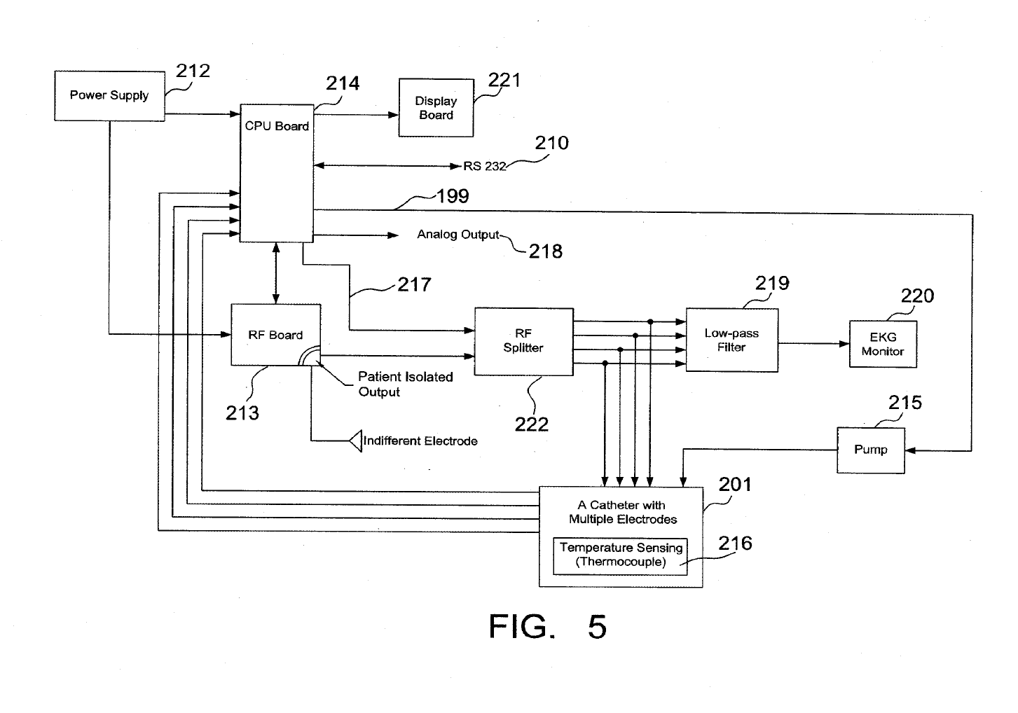

FIG. 4 is a system installation diagram of an RF ablation system with an irrigated ablation catheter. The system includes a catheter 201 with multiple electrodes, a connecting cable 202, an RF generator 203, an EKG connecting cable 204, and a DIP (Dispersive Indifferent Patch) electrode device 205 that is connected to the RF generator 203 through an isolated patient connector 208. The DIP electrode device 205 is placed under a patient, during an ablation procedure, to provide a closed-loop circuit of the RF energy delivery system. The catheter 201 has a plurality of electrodes 206 and a plurality of temperature sensing elements. Each temperature sensing element is located at the proximity of each of the electrodes 206. The catheter 201 is connected to the RF generator 203 through the connecting cable 202. Each of the insulated temperature wires and the conducting wires of the catheter 201 are secured to a connector 207 contact pin of the catheter 201. Therefore, the measured temperature data from each of the multiple electrodes is relayed to a control mechanism located in the CPU board 214 (FIG. 5) of the RF generator 203. In the meantime, the RF energy output is delivered through each of the conducting wires to a respective individual electrode on the catheter 201. The control mechanism of the CPU board 214 also controls the operation of an irrigation pump 215 which is used to pump irrigation fluid to the irrigated catheter 201.

At the back panel of the RF generator 203, there are a power supply port 209, a data output port 210, and a pump port 199. An optional footswitch 211 is also provided for the user's convenience. Either the footswitch 211 or a button 238 on the front panel of the RF generator 203 can be used to start and stop the RF energy delivery.

FIG. 5 is a block diagram of the RF ablation system of FIG. 4, to provide RF energy delivery through an RF splitter to each of the multiple electrodes of the ablation catheter 201. The power supply source 212 is connected to the RF generator 203 having the RF board 213 and the CPU board 214. A software program becomes an integral portion of the CPU board 214. A catheter 201 that has multiple electrodes has a plurality of temperature sensing elements 216. Each temperature sensing element 216 is associated with one of the electrodes 206. The measured temperature data is relayed to the software program inside the CPU board 214. The data from the CPU board 214, such as power, temperature, impedance, and time, is then displayed via a display board 221. The command or instruction is issued from the CPU board 214 to the RF board 213 to control the RF energy output. An RF splitter 222 is employed to split the RF energy in order to deliver it to one or more of the conducting wires, wherefrom thereafter the RF energy output is relayed to the corresponding electrode or electrodes. A digital control signal 217 from the CPU board 214 to the RF splitter 222 controls the manner in which the RF energy is delivered to the one or more conducting wires. The RF energy may be delivered in an independent manner, or a sequential manner, or a simultaneous manner. Data can be stored in the CPU 214 or outputted through an RS232 port 210 to an external computer 198 (FIG. 4) for data analysis. Data may also be outputted to an analog output port 218. The CPU board 214 sends a control signal via the pump port 199 to the pump 215 to control the operation of the pump 215, such as, for example, the flow rate of the fluid delivered by the pump 215 to the irrigated catheter 201.

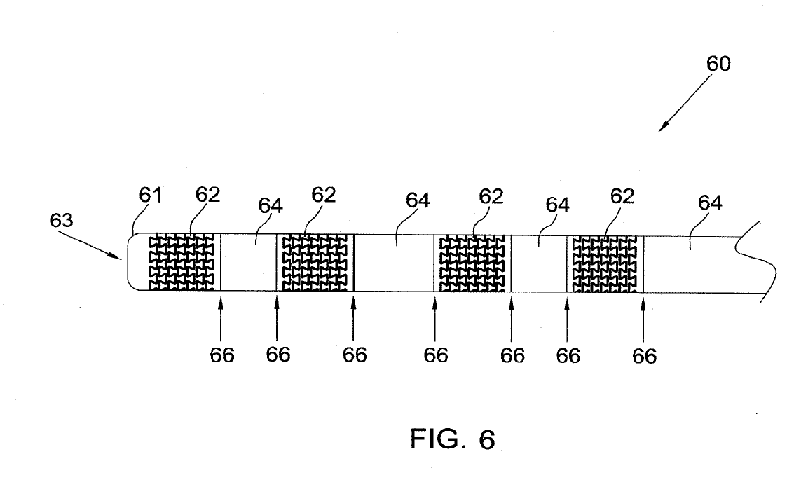

The distal portion of the catheter may have a variety of configurations. FIG. 6 shows an example of a distal portion of an irrigated ablation catheter. The distal portion has a tip electrode 61 at the distal end 63. The catheter 60 includes flexible ring electrodes 62 having gaps cut into a cylindrical sheet to allow fluid to flow out. One of the flexible ring electrodes 62 also forms the tip electrode 61. For example, elution holes in fluid communication with a fluid lumen via ducts are provided in a portion of the elongated body underneath the flexible ring electrodes 62, and the fluid flows through the elution holes and the gaps in the electrodes 62. The gaps may be laser cut into the cylindrical sheets of the electrodes 62. The flexible ring electrodes 62 are spaced from the proximal end of the elongated body by an electrically nonconductive segment 64, and the electrodes 62 are spaced from each other longitudinally by electrically nonconductive segments 64. An edge 66 is formed between an electrode end of the segmented ablation electrode 62 and a nonconductive segment end of the electrically nonconductive segment 64. Examples of flexible ring electrodes with elongated gaps can be found, for example, in US2008/0294158 and W0/2008/147599, the entire disclosures of which are incorporated herein by reference.

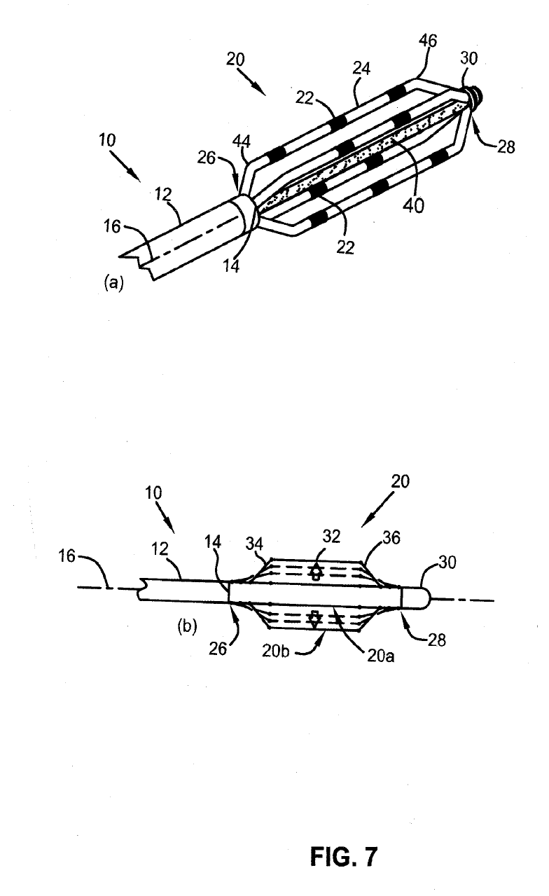

FIG. 7 shows another example of a distal portion of a catheter in the form of an assembly of staggered ablation elements. In the perspective view of FIG. 1a, the ablation catheter 10 includes an elongated catheter body 12 extending longitudinally between a proximal end (not shown) and a distal end 14 along a longitudinal axis 16. An ablation element assembly 20 includes a plurality of ablation elements 22 connected to the catheter body 12. The ablation elements 22 are discretely spaced from each other longitudinally and/or laterally, and at least two of the ablation elements 22 are spaced from one another longitudinally.

In FIG. 7, the ablation elements 22 are electrodes such as RF electrodes. The ablation electrode assembly 20 is connected to the distal end 14 of the catheter body 12. As seen in FIGS. 7a and 7b, the electrode assembly 20 includes a plurality of spines 24, which may be oriented generally longitudinally. Each spine 24 has a proximal end 26 connected to the catheter body 12 and a distal end 28. The distal ends 28 of the spines 24 are connected to a spine distal junction 30. Each spine 24 includes an intermediate segment 32, a proximal stiffness change between the proximal end 26 and the intermediate segment 32 of the spine 24, and a distal stiffness change between the distal end 28 and the intermediate segment 32 of the spine 24. The spines 24 include a plurality of ablation electrodes 22 on the intermediate segments 32. As shown in FIG. 7b, the electrode assembly 20 is movable between a collapsed arrangement 20a and an expanded arrangement 20b with the intermediate segments 32 of the spines 24 in the expanded arrangement 20b moving outwardly relative to the proximal ends 26 and distal ends 28 of the spines 24 with respect to the collapsed arrangement 20a. In use, the catheter 10 with the electrode assembly 20 is inserted into a vessel of a patient in the collapsed arrangement 20a (inside a guiding sheath or the like) and deployed into the expanded arrangement 20b. The ablation electrodes 22 in the expanded arrangement 20b contact surfaces to be ablated to ablate tissue and/or denervate nerves. A longitudinal rod 40 in the center of the electrode assembly 20 is connected to the spine distal junction 30, and can be used to pull the spine distal junction 30 toward the distal end 14 of the catheter body 12 to move the electrode assembly 20 toward the expanded arrangement 20b. Additional examples of an assembly of ablation elements can be found in US2011/0118726, the entire disclosure of which is incorporated herein by reference.

In other embodiments, a collapsible balloon instrumented with electrodes instead of the basket may be used to apply RF energy or the like for denervation. Other examples include a deployable needle or needles, with or without chemical injection, a heated balloon, and a chemical agent denervation balloon. The catheter may be a cryogenics catheter which uses a mixed-gas Joule-Thomson refrigeration unit to cool the tip of a catheter. The catheter may be a laser energy ablation catheter coupled to a laser generating and control system. The catheter may include an ultrasonic or acoustic device to direct ultrasonic or acoustic energy to denervate nerves. The catheter may include a mechanical device to cut nerves.

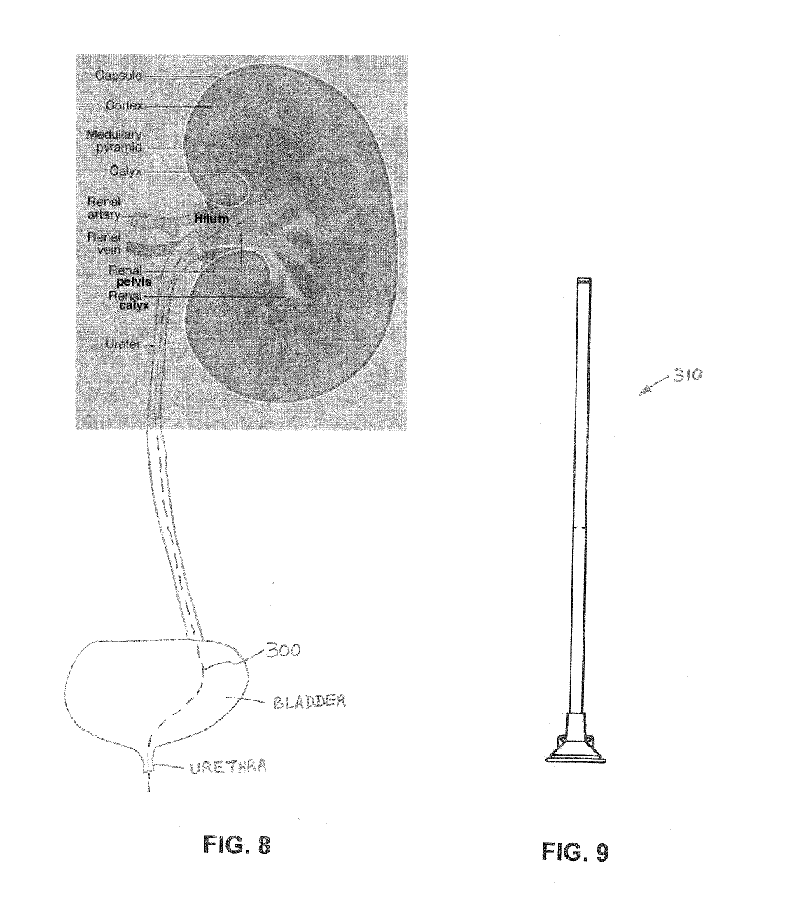

The catheter can be used for renal afferent denervation in the renal pelvis. The catheter is introduced through the urethra, bladder, and ureter, and advanced toward the kidney to reach the renal pelvis. FIG. 8 shows a path in the form of a broken line 300 through the urethra, bladder, and ureter to introduce the distal portion of the catheter to the renal pelvis and, in some case, to the renal calyx. In males, the catheter is inserted into the urinary tract through the penis. In females, the catheter is inserted into the urethral meatus. A guidewire may be used to facilitate the delivery of the catheter. Guidewires are often passed into the uretheral orifice cystoscopically and are then directed into the renal pelvis with fluoroscopic imaging guidance. A dilator or sheath may also be used to facilitate passage of the catheter. Use of a guidewire allows for multiple passes of the instrument while maintaining access to the upper urinary and the kidney. The use of a uretheral sheath minimizes trauma to the uretheral meatus and intramural ureter. FIG. 9 shows an example of a uretheral sheath 310. It is typically a flexible, hydrophilic coated, reinforced polymer sheath. The sheath 310 is inserted transurethrally into the ureter over a guidewire.

After introducing the catheter into the renal pelvis cavity surrounded by the renal pelvis wall, with radio-opaque markers and the fluoroscope imaging guide, the catheter can be maneuvered and deflected to target any and all areas of the renal pelvis for renal denervation, for directly denervating the afferent nerves in the renal pelvic regions. Possible targeted afferent denervation sites include the renal pelvis and the renal calyxes, particularly the major calyxes. One method of denervation is to create lesions to block the afferent nerves in the renal pelvis or the renal calyx. This is done by direct renal afferent denervation on the renal pelvis or the renal calyx using RF or any of the energy sources described above. In addition, due to the close proximity of the renal vessel (artery or vein) with respect to the renal pelvis, selective renal arterial nerve denervation of the renal nerves in the renal vessel can be achieved via the renal pelvis using an appropriate energy form such as HIFU (high intensity focused ultrasound), for example. The denervation procedure may involve bilateral renal denervation. After completion of afferent denervation of one kidney, the distal portion of the catheter will be withdrawn back into the bladder and then advanced into the other kidney via the other ureter to perform renal afferent denervation.

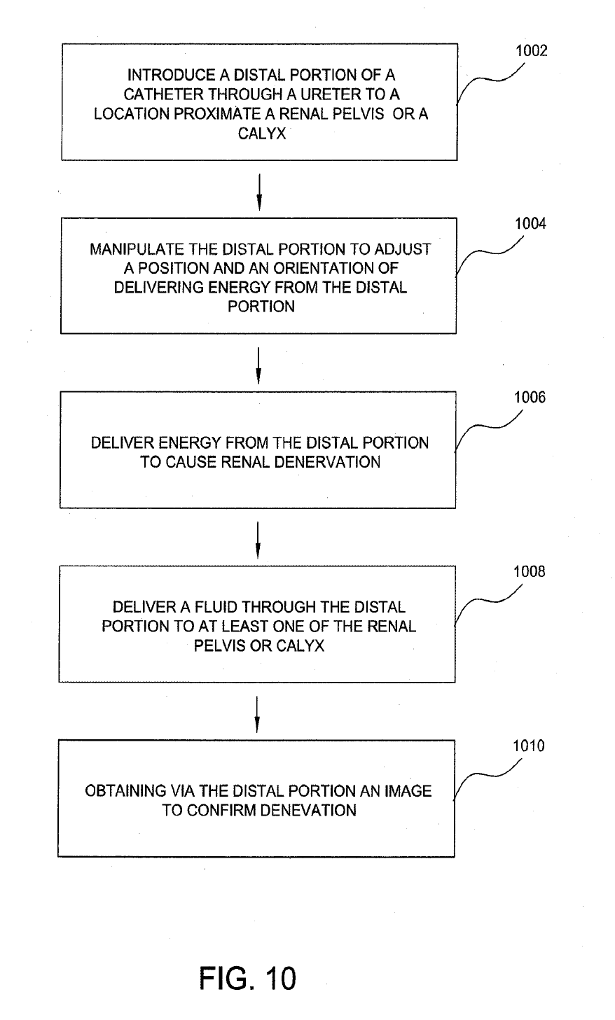

FIG. 10 shows an example of a flow diagram illustrating a renal denervation process according to an embodiment of the invention. In step 1002, a distal portion of a catheter is introduced through an interior of a vessel (such as the ureter) of a patient to a location at or proximate one of a renal pelvis or a calyx (sufficiently close to deliver energy to cause renal denervation). In step 1004, the distal portion is manipulated through a control member (e.g., handle) disposed near the proximal end of the catheter to adjust a position and an orientation of delivering energy from the distal portion. In step 1006, energy is delivered from the distal portion to cause renal denervation. This may involve denervating at least some tissue proximate at least one of the renal pelvis or the calyx or a renal vessel. For example, denervation of the renal pelvis may involve denervating at least some tissue adjacent the renal pelvis wall of the renal pelvis. This may be achieved via contact between the distal portion and the renal pelvis wall. During the denervation of step 1006, a fluid may be delivered via the catheter lumen through the distal portion to at least one of the renal pelvis or the calyx in step 1008. Step 1010 involves obtaining via the distal portion an image of denervation of the at least some tissue.

The systems and methods described herein can be used to treat not only hypertension, but chronic renal diseases, cardiovascular disorders, cardiac arrhythmias, and clinical syndromes where the renal afferent activation is involved. The procedure is easy to perform and familiar to nephrologists. Due to the smaller size of the catheter in preferred embodiments as compared to uretheral endoscopy and surgical instrumentation, this can be a much easier and safer procedure.

In the description, numerous details are set forth for purposes of explanation in order to provide a thorough understanding of the present invention. However, it will be apparent to one skilled in the art that not all of these specific details are required in order to practice the present invention. Additionally, while specific embodiments have been illustrated and described in this specification, those of ordinary skill in the art appreciate that any arrangement that is calculated to achieve the same purpose may be substituted for the specific embodiments disclosed. This disclosure is intended to cover any and all adaptations or variations of the present invention, and it is to be understood that the terms used in the following claims should not be construed to limit the invention to the specific embodiments disclosed in the specification. Rather, the scope of the invention is to be determined entirely by the following claims, which are to be construed in accordance with the established doctrines of claim interpretation, along with the full range of equivalents to which such claims are entitled.

What is claimed is:

1. A method for denervation, comprising:
introducing a distal portion of a catheter through an interior of a vessel of a patient to a location at or proximate one of a renal pelvis or a calyx, the catheter including an elongated catheter body extending longitudinally between a proximal end and a distal end, the catheter body including the distal portion at the distal end and a catheter lumen from the proximal end to the distal end; and
delivering energy from the distal portion to denervate at least some tissue proximate at least one of the renal pelvis or the calyx.

2. The method of claim 1,
wherein the distal portion of the catheter is introduced through a ureter of the patient.

3. The method of claim 2,
wherein the distal portion of the catheter is introduced through a urethra and a bladder of the patient.

4. The method of claim 3, further comprising:
inserting a uretheral access sheath through the urethra into the ureter to facilitate introduction of the distal portion of the catheter through the urethra and the ureter.

5. The method of claim 1,
wherein energy is delivered from the distal portion to cause renal afferent denervation of at least some tissue proximate at least one of the renal pelvis or the calyx.

6. The method of claim 1,
wherein the energy delivered is selected from the group consisting of radio-frequency (RF) energy, electrical energy, laser energy, ultrasonic energy, high-intensity focused ultrasound (HIFU) energy, cryogenic energy, thermal energy, chemical energy, and mechanical energy.

7. The method of claim 1, further comprising:
delivering a fluid via the catheter lumen through the distal portion to at least one of the renal pelvis or the calyx.

8. The method of claim 1, further comprising:
manipulating the distal portion through a control member disposed near the proximal end of the catheter to adjust a position and an orientation of delivering energy from the distal portion.

9. The method of claim 1, further comprising:
obtaining via the distal portion an image of denervation of the at least some tissue.

10. A method for denervation, comprising:
introducing a distal portion of a catheter through an interior of a vessel of a patient to a location at or proximate a renal pelvis, the catheter including an elongated catheter body extending longitudinally between a proximal end and a distal end along a longitudinal axis, the catheter body including the distal portion at the distal end and a catheter lumen from the proximal end to the distal end; and
delivering energy from the distal portion at the location at or proximate the renal pelvis to cause renal denervation.

11. The method of claim 10,
wherein energy is delivered from the distal portion at the location at or proximate the renal pelvis to cause renal denervation in at least one of the renal pelvis or a renal blood vessel.

12. The method of claim 10,
wherein energy is delivered from the distal portion to cause renal afferent denervation of at least some tissue proximate at least one of the renal pelvis or a renal vessel.

13. The method of claim 10,
wherein the energy delivered is selected from the group consisting of radio-frequency (RF) energy, electrical energy, laser energy, ultrasonic energy, high-intensity focused ultrasound (HIFU) energy, cryogenic energy, thermal energy, chemical energy, and mechanical energy.

14. The method of claim 10, further comprising:
delivering a fluid via the catheter lumen through the distal portion to the renal pelvis.

15. The method of claim 10, further comprising:
manipulating the distal portion through a control member disposed near the proximal end of the catheter to adjust a position and an orientation of delivering energy from the distal portion.

16. The method of claim 10,
wherein the distal portion of the catheter is introduced through a urethra, a bladder, and a ureter of the patient.

17. A method for denervation, comprising:
introducing a distal portion of a catheter through an interior of a vessel of a patient to a location at or proximate a renal pelvis, the catheter including an elongated catheter body extending longitudinally between a proximal end and a distal end, the catheter body including the distal portion at the distal end and a catheter lumen from the proximal end to the distal end; and
delivering energy from the distal portion at the location at or proximate the renal pelvis to denervate at least some tissue proximate the renal pelvis.

18. The method of claim 17,
wherein energy is delivered from the distal portion at the location at or proximate the renal pelvis to denervate at least some tissue adjacent a renal pelvis wall of the renal pelvis.

19. The method of claim 17,
wherein energy is delivered from the distal portion via contact between the distal portion and a renal pelvis wall of the renal pelvis.

20. The method of claim 17,
wherein the distal portion of the catheter is introduced through a urethra, a bladder, and a ureter of the patient.
